(19) United States
(12) Patent Application Publication
(10) Pub. No.: US 2015/0272806 A1
(43) Pub. Date: Oct. 1, 2015

(54) SHAPE MEMORY ELEMENT FOR MEDICAL DEVICES

(71) Applicant: Bauerfeind AG, Zeulenroda-Triebes (DE)

(72) Inventor: Hans B. BAUERFEIND, Zeulenroda-Triebes (DE)

(73) Assignee: Bauerfeind AG

(21) Appl. No.: 14/739,494

(22) Filed: Jun. 15, 2015

Related U.S. Application Data
(63) Continuation of application No. 13/822,968, filed on Apr. 16, 2013, now abandoned.

(30) Foreign Application Priority Data
Oct. 4, 2010 (DE) 10 2010 048 083.5

(51) Int. Cl. A61H 1/00

(52) U.S. Cl. CPC A61H 1/008; A61F 2002/7818

(57) ABSTRACT

The invention relates to medical devices, in particular body support bandages and orthoses, for the human or animal body having at least one element, which generates or provides supporting force, compression or introduction of pressure, and which comprises or consists of shape memory material.

### REFERENCE TO RELATED APPLICATION

This is a continuation application of Ser. No. 13/822,968, filed Apr. 16, 2013 currently pending. The subject matter of the aforementioned prior application is hereby incorporated herein by reference.

### BACKGROUND OF THE INVENTION

The invention relates to medical devices for a human or animal body, and in particular bandages, having at least one element which generates or provides supporting force, compression or introduction of pressure, and which comprises or consists of shape memory material.

Medical devices such as bandages or orthoses, which can be applied to parts of the body in order to support these body parts, in particular through specific effective forces, or in order to exert compression on those body parts, are known from the prior art. Such devices are primarily used in the field of phlebological therapy or prophylaxis, as well as in orthopedic therapy or prophylaxis, especially for the improvement or maintenance of body functions in parts of the body such as limbs. Devices known from the prior art are designed in the form of a stocking or a belt, and generally completely enclose the body part in order to provide a supporting function, compression or local introduction of pressure. These devices known from the prior art are primarily designed in the form of bandages or stockings comprising an elastic material or material structure, in particular an interlaced or knitted textile. In particular, one or more elastic elements are incorporated into the textile fabric in a conventional manner, for example in the form of filler threads, wherein said elements are stretched when the device is in an applied state, and which introduce force, support or compression of the body part. Such elements may also be designed in the form of braces, bars or bands inserted into the device, or in the form of pads. Braces, bars or bands, for example, locally reduce the elasticity or mobility of the device when applied in order to enable a supporting or compression function. Pads effect local introduction of pressure or padding. In medical devices known from the prior art, braces, bars and pads are not always well adapted or adaptable to the individual anatomical conditions of the body of wearer. In particular in the case of common, mass-produced products, it has not heretofore been possible to achieve well customized conformation. As in that described above, heretofore devices such as orthopedic shoe inserts have also been insufficiently individually adjustable to the anatomical shape of the wearer. The invention also relates to prostheses and specific parts of prostheses, which conform to the body, in particular a stump. An element according to the invention can thereby also be designed as a prosthetic liner.

In the case of medical devices in the form of bandages designed as a stocking which must exert a high degree of compressive pressure on the body part when in an applied state for support or compression, the fact that said bandages are difficult to apply to the body has proven to be a disadvantage in handling. The application requires that prominent structures such as body joints, for example the heel or elbow, be overcome. This usually requires adequate elasticity of the material. It is thus often not possible to achieve an efficient supporting or compression function. Specifically, phlebological stockings must be pre-expanded prior to application under tension, and then held under tension even during application in order that the stockings can be applied at all.

The demand is also placed on medical devices that these devices be in contact with the body part in such a way that, when applied, they cannot slip off of the body part. This is known to be achieved through a high material tensile strength in conjunction with a knitted contour which is precisely adapted to the body shape. Further, it is known that the devices can be provided with a specific material feature on the side facing towards the body, which counteracts slipping by way of the high coefficient of friction generated there. The problem here is that all of these measures make applying such devices known from the prior art more difficult. A device that exhibits an improved supporting effect, compression effect or pressure introduction properties in an applied state, but can be applied easily and securely, is therefore desirable.

### SUMMARY OF THE INVENTION

The technical problem underlying the invention is in the provision of an improved medical device of a known kind, in particular a bandage or a stocking for the support or compression of a body part, which overcomes the above mentioned drawbacks. In order to solve this technical problem, a bandage is proposed comprising at least one element that generates or provides supporting force, compression and/or local introduction of pressure, wherein the element comprises, or preferably consists of, at least one material having shape memory characteristics, meaning one or more shape memory materials.

Accordingly, the subject matter of the invention is a bandage that provides a supporting or compression effect, which can be applied to an animal or human body, comprising or consisting of a knitted or interlaced textile having at least one element that generates a supporting force or compression, wherein the element comprises or consists of shape memory material, wherein in the applied state, a programmed shape transition can be triggered in the shape memory material through the effects of body heat from the body. This is made possible according to the invention in that the bandage can be converted or is converted from a (first) expanded form, which facilitates the application of the bandage to the body, to a (second) contracted form, which provides the supporting or compression effect of the bandage to the body.

For the purposes of the invention, a “medical device” is understood to refer to bandages that ensure the success of the medical treatment by exerting a compensating, supportive or relieving effect externally on the body, or that make it possible to overcome physical disabilities. These include body replacement parts (prostheses) and parts thereof such as prosthetic liners, and phlebological and orthopedic devices, in particular bandages, orthoses, compression stockings, orthopedic shoe inserts and the like. For the purposes of the invention, on the other hand, a “medical device” is not understood to refer to visual devices (glasses, contact lenses), nor technical products that are intended for internal use in the body (implants, stents, probes), or that serve to introduce drugs or other therapeutics for internal use in the body (syringes, inhalers, drug delivery devices).

In this case, a material having shape memory characteristics, that is to say a shape memory material, is understood to be a material, a material compound or composite that undergoes a shape transition from a first defined, and in particular temporary, shape into a second defined, and in particular temporary or, alternatively, permanent shape, triggered by an external factor, for example an input of energy through an increase in temperature. In a particular embodiment, the shape transition is reversible or quasi-reversible.

The shape transition may take the form of a change in at least one dimension of the outer measurements of the materials and/or the three-dimensional shape thereof. Alternatively or in addition, this shape transition is characterized by a change in at least one physical property of the material, in particular in a change in the modulus of elasticity. By modifying the modulus of elasticity, the supporting or compressing effect of the element according to the invention provided within the device according to the invention can be adjusted in a controlled manner.

In an alternative or additional embodiment, the shape transition is characterized as a change in the specific heat capacity of the material. This is especially applicable when additionally or exclusively heat-dissipating or heat-insulating effects are to be triggered with the device according to the invention.

In particular, the invention differs from so-called “thermofoams” known from the prior art, which do not constitute shape memory materials within the meaning of this invention. These polymers having a strongly temperature-dependent material hardness and elasticity lack the characteristic of taking on at least one other stable temporary shape in addition to a specific permanent shape, which is only lost through the action of an external factor.

Here, a shape memory material is understood to be an alloy, a mixture and/or a compound or composite material or fabric comprising multiple, in particular different shape memory materials. In a particular embodiment, the alloys, mixtures, compounds, composites or fabrics of the shape memory materials are shaped in such a way that such shape memory materials used therein feature distinctively shaped shape transitions, preferably having different transition conditions, which are preferably initiated by a variety of external factors. The bringing together of such different materials provided in this embodiment creates a material that exhibits expanded shape memory characteristics. Thus, in particular, additional shape transitions can be achieved. A second shape transition can be designed as a “reset” of the second shape into the first shape in order to allow quasi-reversible shape transitions.

In a particular embodiment, the material of the element according to the invention is formed from a first shape memory material, which is specifically designed to undergo a first shape transition from a first defined shape into a second defined shape in the presence of a specific first external factor, and is formed with at least one second or addition shape memory material, which is specifically designed to undergo an additional shape transition in the presence of another external factor. In a particular variant, the first factor for the induction of the first shape transition of the first shape memory material differs from the second factor for the induction of the second shape transition of the additional shape memory material. In particular the quality/quantity of the first factor differs from the quality/quantity of the second factor. In particular, the first shape transition of the first shape memory material has a shape that differs from the second shape transition of the second or additional shape memory material. Composite-materials, in particular, can be designed in such a way that they can be reversed and “switched” into two different forms or shapes; hysteresis is made possible by the two different external factors of the shape transitions.

In a further embodiment, the shape memory materials are used so that they are separated from one another in the device. They are designed in the device especially as counteracting actuators, in the manner of flexor and extensor, in order to allow quasi-reversible shape transition of the device as a whole from a first shape into a second shape and back.

Shape memory materials per se are known from the prior art. The embodiments of shape memory materials suitable for the present invention are mainly shape memory polymers (SMP) or, where appropriate, shape memory alloys (SMA), and modifications thereof. Shape memory materials comprise so-called “switching segments”, which are present in the polymer in covalent and non-covalent cross-links. External influence on the switching segments through physical or chemical stimuli (external factors) leads to a change in the structure of the material, which takes the form of a change in the external shape and dimensions (shape change in the strict sense) and/or in changes in the physical properties, for example of the modulus of elasticity (shape change in the broader sense). Shape memory materials generally comprise a so-called permanent first shape and a temporary second shape that is imprinted on it through appropriate measures, that is to say “programmed”. As a rule, the shape transition from the temporary second shape into the permanent first shape is irreversible in such shape memory materials. By way of appropriate combination of alloys, compound materials and composites of a number of different shape memory materials or shape memory materials and switching elements having different characteristics, as provided for elsewhere in the context of the present invention, an induced shape transition is triggered, which has at least two different predefined, temporary shapes, and which is quasi-reversible.

In a preferred embodiment, the shape memory material is a shape memory polymer. Appropriate shape memory materials are described in WO2007/104757 A1, in particular formulas Ia to Ic and IIa to IIc as well as FIGS. 1 to 5. In particular, the shape memory polymer is selected from the group of SMPs consisting of: polyesters, polycarbonates, polyethers, polyamides, polyimides, polyacrylates, polyvinyls, polystyrenes, polyurethanes, polyether urethanes, polyetherimides, polymethacrylates, polyoxymethylene, poly-ε-caprolactone, polydioxanone, silicones, natural rubbers, synthetic rubbers, and mixtures and compositions thereof. In an embodiment of the invention, the shape memory material is a polyurethane elastomer, alone or in combination with one of the aforementioned materials. In another embodiment, the shape memory material is a polyisoprene, alone or in combination with one of the aforementioned materials. In another embodiment, the shape memory material is a styrene-isoprene-butadiene block copolymer, alone or in combination with one of the aforementioned materials.

The elements having shape memory characteristics are produced in a conventional manner. Measures known in the prior art include injection molding, extrusion, spinning, drawing, blowing and/or thermoforming from semi-finished products. Other production methods, which are especially suitable for the formation of the element in a device comprising a textile material such as bandage, include casting and direct coating in particular. In addition, preformed or premolded elements can also be applied to the textile fabric through welding (high-frequency welding) or bonding. Further known methods are also applicable.

The external factor is especially selected from a physical and a chemical stimulus. In particular, the physical stimulus is an input of energy selected from thermal radiation, contact heat, convection, induction, electromagnetic radiation, exposure to ultrasound, electrical current, and electrical voltage change as well as combinations thereof. Alternatively or additionally, the external factor is an effect by a chemical agent, which can interact with functionalizations in the shape memory materials, as well as combinations of the physical and/or chemical factors with one another.

In a preferred embodiment, the external factor is a temperature increase above a “switching temperature” which is specific to the shape memory material. The specific “switching temperature” can be modified in the shape memory material and selectively adjusted through suitable chemical measures, for example plasticizers etc. In a special variant, an external factor that can trigger a shape transition is body heat, in particular an increase in temperature to 28° C. or more, or 32° C. or more.

In another variant, the temperature increase is triggered by hot air or heat radiation (heat lamp). In this case, the temperature increase is preferably 40° C. or more, or 45° C. or more, at least temporarily.

In an additional variant, an agent that generates or produces an external factor, for example in response to a mechanical or electric stimulus, is introduced into the medical device according to the invention, wherein said factor triggers the shape transition in the shape memory material. To this end, the agent and the shape memory material are preferably disposed adjacent to one another. For example, frictional heat may be generated, which then triggers the shape transition in the shape memory material. Alternative variants provide for the release of a chemical heat of reaction in the agent.

In other or additional embodiments, an external factor is the change in the moisture level in the medium surrounding the shape memory material, in particular in the surrounding air. This can be selectively achieved by adding steam or water. In a special variant thereof, an external factor that can trigger a shape transition is the moisture level in proximity to the body, in particular in the case of skin contact, especially 70% relative humidity or more, or 90% relative humidity or more.

In an alternative or additional embodiment, the external factor is a chemical “stimulus” in the form of a change in the composition of the medium surrounding the shape memory material. A special variant thereof is a change in the pH value of the surrounding medium. It is thus provided that the shape memory material, and in particular the entire device, be brought into contact with an aqueous solution or mist, which has a specific pH value that interacts with the functionalized shape memory material. In a special variant thereof, a change in shape can be produced through contact with an alkaline aqueous solution, especially pH 8 or more.

In a special variant thereof, an external factor that can trigger a shape transition is the pH value of the skin of the body, especially pH 6 or less or pH 5 or less. The acidic pH value of the body can deploy the effect in the functionalized shape memory material, especially in conjunction with body moisture.

In a special embodiment of the invention, the shape memory materials of the element providing the supporting force, compression or local introduction of pressure are designed in such a way that they exist in the first defined, temporary shape when the device is in the stored state, which is distinguished by a high degree of elasticity and/or an expanded form, for example, whereby especially the application of the device can be facilitated. The transition into the second defined shape is triggered in the applied stage, caused by body heat or the pH value of the skin as an external factor for example, wherein that second shape is especially distinguished by a reduced elasticity and/or a contracted form. Such an embodiment of the device according to the invention, in particular in an embodiment as a bandage or stocking, is advantageously flexible and loose when being applied, and firmly positions itself in an improved manner on the body part after application due to the shape transition, which is triggered here by an external factor in order to exert an improved supporting or compression effect, or to provide an improved local introduction of pressure. In the case of an embodiment such as a prosthetic liner, a better fit, and therefore an improved hold of the prosthesis on the stump, can be achieved while at the same time achieving more comfortable handling during application.

In a special embodiment of the invention, if is provided that the user/wearer “programs” the device, in particular the bandage, in a first temporary shape before wearing, for example by exposing it to a physical or chemical factor, and can easily apply the device in this conformation. In an applied state, the second, and in particular permanent, shape of the material is “recalled”, by exposing the shape memory material to a (further) external factor. Thus this embodiment provides that the device in the applied state is “programmed” in a conformation that is prepared for application on the body using the shape memory material. The programming can be done by stretching the removed device on a preformed support and then exposing it to an external factor, for example the effects of heat such as hot air, for example in a tumble dryer, or by immersion in an alkaline detergent solution or an acidic solution.

Pre-expanding of the device before applying it and applying it under tension are unnecessary. The supporting function, the compressive pressure and/or the local introduction of pressure are generated according to the invention by recalling a second temporary shape or the permanent shape of the shape memory material for the elements in an applied state. In this context, the invention advantageously allows the “compression shape” of the applied device to be recalled in specific zones, for example in the embodiment as a compression stocking, in order to achieve a customized conformation of the compression effect depending on the anatomical situation, indication of treatment and/or therapeutic objective. In an exemplary, simple embodiment, this can be accomplished first and foremost by a locally limited application of steam, hot air or infrared radiation to the element according to the invention in order to create local and customized zones of increased supporting or compression effect, or an introduction of pressure where necessary.

In this aforementioned embodiment, when the device is in its functional conformation applied to the body, the shape memory material exists in its permanent shape, and when the bandage is in a removed state in the conformation prepared for application, the shape memory material exists in its temporary shape. Naturally in the context of the invention, the reverse situation of permanent and temporary programmable conformations is also provided.

In an embodiment of the invention, the element creating the supporting force is a filler thread in the textile fabric of the medical device, in particular in the fabric of the bandage. In an alternative embodiment, the element is a weft in the fabric of the bandage.

in one embodiment, the element creating the supporting force is a support brace or bar, for example a support band that is inserted in the bandage and, in particular, that is connected in a force-fitting manner with said bandage.

In one embodiment, the element creating the supporting force is a pad, which is inserted or incorporated into the device or the bandage, wherein the material having shape memory characteristics is the pad. In a specific variant, the pad comprises the material having shape memory characteristics. This allows an improved selective introduction of pressure, that is to say, adapted to the individual anatomical conditions prevailing in the wearer. In this embodiment, the invention provides, in particular, that through programming, a pad which is mass produced in a general shape can be brought into a temporary shape that is customized to the wearer. This can be done prior to wearing by measuring the body part (e.g. contactless digital body measurement) and corresponding programming of the pad for the calculated physical anatomy.

Programming can be performed based on the specific indication of treatment, the therapy or the therapy plan. Thus a plurality of different products that are adapted to the anatomy and/or therapy or therapy plan can be economically produced based on one or a few mass-produced devices such as orthoses or bandages, while also achieving improved efficacy. Conversely, a first temporarily programmed shape of the pad favoring the application of the bandage can be converted into a permanent second shape better adaptable to the physical anatomy when wearing, by triggering the shape transition. This improves the fit of the device or the bandage and the introduction of pressure via the pad.

in one embodiment, the element comprising or consisting of shape memory material, the pad or support brace in the device is formed so that it is stiffer and less elastic in an early phase of therapy, in particular in order to allow the supporting effect, guidance or introduction of pressure to the body, for example to the joints. In a later phase of the therapy, the element can be converted into a second shape by triggering the shape transition, which is especially characterized by a greater elasticity. The element then takes on a cushioning or damping function. In this embodiment, it is especially provided that the first shape of the shape memory material of the element is the programmed temporary shape, and the second shape is a permanent shape, and reversibility into the first programmed shape need not exist.

The invention also includes the use of the shape memory materials described herein as a component of elements providing a supporting effect, compression or local introduction of pressure in all kinds of medical devices, and in particular bandages, stockings and orthoses. According to the invention, in addition to phlebological support stockings or compression stockings, these also include joint supports and orthoses, in particular ankle orthoses, knee orthoses, wrist orthoses, shoulder orthoses, orthoses for the spine, in particular lumbar orthoses and neck orthoses. Further, this includes orthopedic inserts and means for foot protection.

An additional application of the invention is in the field of prosthetics. According to the invention, the shape memory material may also improve the fit of a prosthesis to the body. In a special embodiment, the element comprising or consisting of shape memory material is designed as a prosthetic liner. A shape transition in the prosthetic liner can thereby be triggered for example through activation by means of body heat, skin moisture and/or the pH value of the skin, wherein that shape transition establishes a closer connection of the prosthesis with the stump or with the prosthetic stump sock.

In addition, the invention provides for the use of such elements comprising shape memory materials in the areas of sports and lifestyle products as well. This especially includes functional clothing and sports clothing that supports anatomical function, into which local elements of the aforementioned type are inserted or integrated, for example.

The subject matter of the invention is also the use of the medical device according to the invention for the phlebological treatment, prophylaxis or therapy of a human or animal body.

The subject matter of the invention is also the use of the medical device according to the invention for the orthopedic treatment, prophylaxis and therapy of a human or animal body, in particular in the form of an orthosis or an orthopedic shoe insert.

The subject matter of the invention is also the use of the medical device according to the invention for the medical prosthetic restoration of a human or animal body, in particular in the form of a prosthesis or a prosthetic liner.

The subject matter of the invention is also the use of one or more of the shape memory materials characterized above in order to improve the supporting function, the compression function, the conformation to the body and/or the introduction of pressure of a medical device, a body support bandage, in particular a compression stocking of an orthosis, an orthopedic shoe insert of a prosthesis, or a part thereof. Also associated with this is the use of one or more of the shape memory materials characterized above in order to improve handling when applying or removing a medical device, a bandage, and in particular a compression stocking or an orthosis.

The invention is illustrated in greater detail in the following examples and figures, without these being understood to be limiting.

### BRIEF DESCRIPTION OF THE DRAWINGS

The figures show:

FIG. 1A: Detailed section from the textile fabric of a compression stocking having filler threads comprising shape memory material;

FIG. 1B: Compression stocking having filler threads comprising shape memory material;

FIGS. 2A and 2B: Schematic representation of an applied compression stocking (FIG. 2A), which is applied to the physical shape of the leg in a zonally selective manner (FIG. 2A) and which effects the controlled compression (FIG. 2B) as a result of the shape transition, triggered after application, of the filler threads comprising shape memory material;

FIG. 3: Reversible or quasi-reversible shape transition of the filler threads comprising shape memory material, triggered by a change in the pH value of the surrounding medium.

### DETAILED DESCRIPTION OF THE INVENTION

### Example

### Compression Stocking

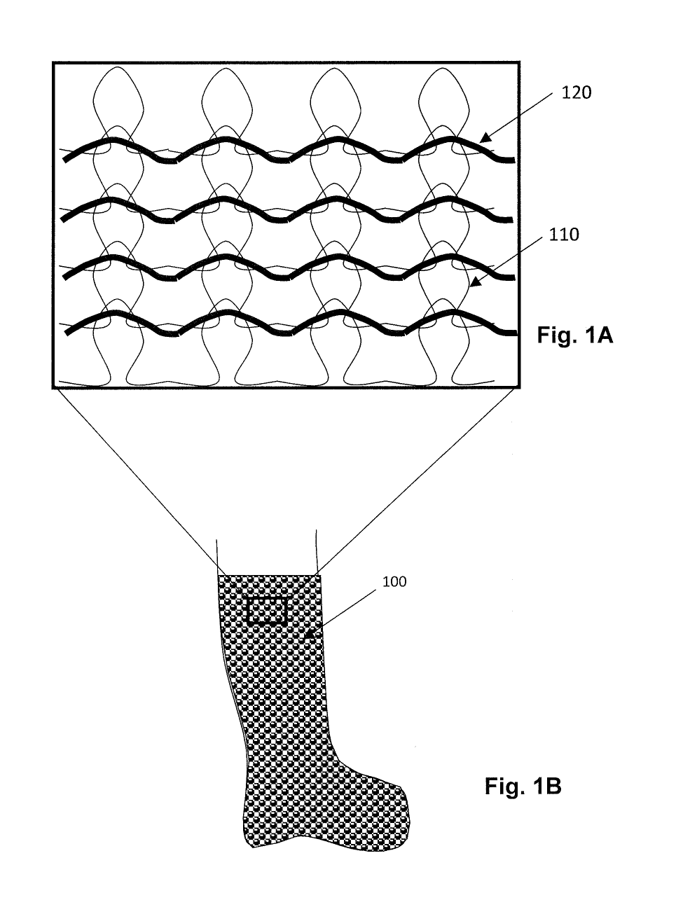

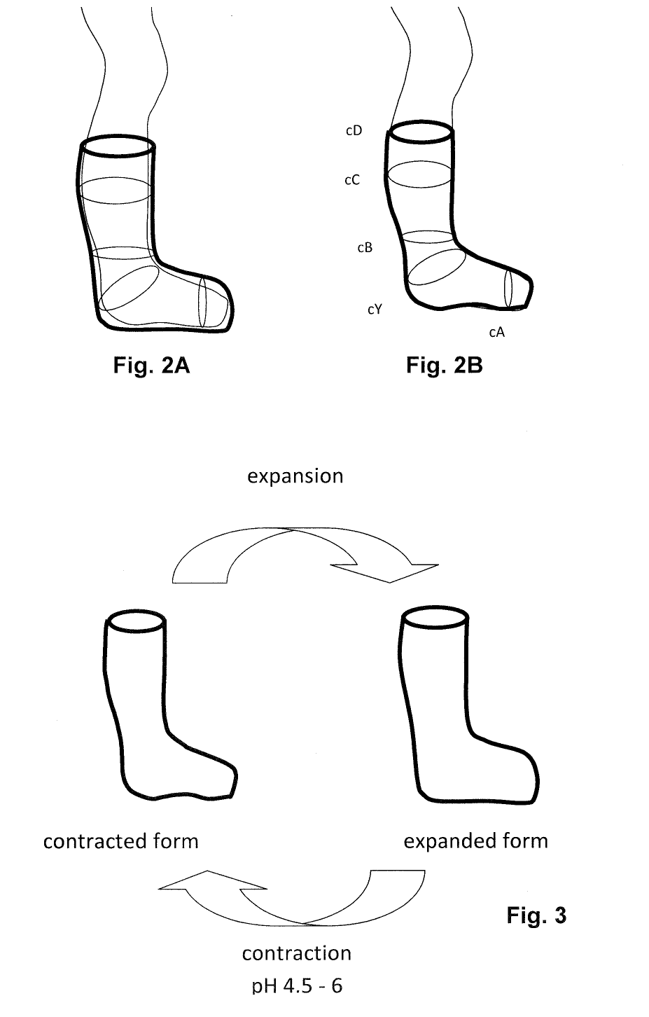

FIG. 1 shows a compression stocking (100) comprising a knitted fabric (110) in connection with a filler thread (120), by way of which the compression can be adjusted in a predefined manner by pre-stretching the thread. The filler thread in this embodiment comprises an acid functional polyurethane. After manufacture, the compression stocking is rinsed in an alkaline bath having a pH value of 8 to 10 and then dried. Corresponding alkaline detergent solutions are known to persons skilled in the art. This results in a reversible elongation of the filler thread, whereby this results in the expanded form of the compression stocking (FIG. 3). In this state, the stocking can be easily applied (FIG. 2A). A defined contraction of the filler thread occurs as a result of contact with the skin having a pH value of 4.5 to 6 (FIG. 2B). This leads to the build-up of the medically indicated compression at the defined measuring points (cD, cC, cB, cY, cA) in accordance with RAL GZ 387, as said points have been bindingly established by the Quality Mark Association for Medical Compression Hosiery. After use, the compression stocking is removed and washed according to the current state of the art. After the wash cycle, the compression stocking is rinsed in an alkaline detergent solution with a pH value of 8 to 10, whereby the stocking is again expanded to the dimensions for application (FIG. 2A). The stocking is then dried as usual. This process can be repeated any number of times over the entire life cycle of the compression stocking (FIG. 3).

1. A bandage for application on the body, comprising an interlaced or knitted textile having at least one element, that generates supporting force and/or compression, comprising shape memory material, wherein in the applied state of the bandage, body heat can trigger a programmed shape transition in the shape memory material, whereby the bandage can be converted from a expanded form that facilitates application of the bandage to the body, to a contracted form that provides the supporting and/or compression effect of the bandage.

2. The bandage according to claim 1, wherein the shape memory material is a compound material, mixture, alloy, compound or composite material comprising multiple materials having different shape memory characteristics.

3. A bandage according to claim 1, wherein the shape transition of the shape memory material is characterized by a change in the outer dimensions thereof.

4. A bandage according to claim 1, wherein the shape transition of the shape memory material is characterized by a change in the modulus of elasticity thereof.

5. A bandage according to claim 1, wherein the bandage is a compression stocking for phlebological treatment.

6. A bandage according to claim 1, wherein the bandage is an orthopedic insert, bandage or orthosis for orthopedic therapy.

7. A bandage according to claim 1, wherein the bandage is a prosthetic liner for a prosthesis.

8. A phlebological therapy method, comprising applying the bandage of claim 1 to a human or animal body in need of phlebological therapy.

9. An orthopedic therapy method, comprising applying the bandage of claim 1 to a human or animal body in need of orthopedic therapy.

10. A medical prosthetic for a human or animal body, comprising the bandage of claim 1.

11. (canceled)
